(19) United States
(12) Patent Application Publication
(10) Pub. No.: US 2007/0106124 A1
(43) Pub. Date: May 10, 2007

(54) Safety check system, method, and program, and memory medium for memorizing program therefor

(76) Inventors: Hiroyuki Kuriyama, Kawasaki (JP); Hideyuki Ban, Kodaira (JP); Futoshi Sagami, Tokyo (JP); Akihiro Omiya, Tokai (JP)

Correspondence Address: REED SMITH LLP, Suite 1400, 3110 Fairview Park Drive, Falls Church, VA, 22042, US

(21) Appl. No.: 11/229,804

(22) Filed: Sep. 20, 2005

(51) Int. Cl. A61B 5/00; G08B 23/00; G08B 1/08

(52) U.S. Cl. 600/300; 340/573.1; 340/539.11

(57) ABSTRACT

A safety check system of the invention is equipped with a safety detection device attached to a body of a checked person, a server for accumulating various pieces of information, and a terminal of a checker for executing a safety check of the checked person, wherein the detection device is equipped with a first radio communication mechanism for making a radio communication with the server, a vital signal detection mechanism for detecting a vital signal of the checked person, and a first control mechanism for creating safety information of the checked person; and the server is equipped with a second radio communication mechanism for making a radio communication with the detection device, a detection history memory mechanism for memorizing a detection history of the checked person, and a second control mechanism for sending the detection history of the checked person in the detection history memory mechanism to the terminal.

### CROSS REFERENCE TO RELATED APPLICATIONS

The present application is based on Japanese application JP 2004-085950, filed on Mar. 24, 2004, the content of which is hereby incorporated by reference into this application.

### BACKGROUND OF THE INVENTION

1. Field of the Invention

The present invention relates to a safety check system of a checked person such as an elder living alone.

2. Description of the Related Art

Recently, accompanied with an increase of an elder living alone, various technologies are devised that take a care of such the elder (checked person) and check safety thereof. For example, it is devised a technology that informs her or his family (checker) of an abnormal detection when measurement data of a vital sensor exceeds a predetermined threshold, making an elder living alone attach to herself or himself the sensor for measuring vital data such as a perspiration amount, a pulse rate, and a skin temperature (see claim 2 and the like in Japanese Patent Laid-Open Publication No. 2003-83590).

The technology disclosed in Japanese Patent Laid-Open Publication No. 2003-83590 is one that notifies the abnormality detection when the acquired data from the vital sensor exceeds the predetermined threshold. But as causes of lack of notification of the abnormality detection, it can be thought that there exist two kinds of causes: 1) there exists no abnormality in a checked person; and 2) the checked person has taken off the vital sensor, a battery of the vital sensor has run out, the vital sensor malfunctions, or there occurs a communication failure.

Accordingly, if a state of lack of notification of the abnormal detection continues, the checker is assured that the checked person lives with no problem; on the other hand, there is no notification for a too long period, there is some case that the checker feels an anxiety that there is no notification due to the cause described in the item 2) (in fact, there occurs some abnormality).

In addition, even if the checker requests an acquisition of safety information of the checked person at arbitrary time, if she or he has exited or falls in a state of the item 2), the checker cannot acquire her or his safety information. In such the state, if the checker cannot acquire the safety information of the checked person at this moment in spite of the checker requesting the safety information again and again, the checker results in further feeling the anxiety.

Here, if the checker can know the reason (battery-running-out of the sensor, communication failure, and the like) why the safety information cannot be acquired, recent safety information of the checked person, creation time of the safety information, the checker can alleviate the anxiety.

Consequently, a safety check system is strongly requested that solves the problem, and can grasp a reason why a checker cannot acquire safety information, time when the safety information is created, and the like, even if a response of the safety information is not acquired.

### SUMMARY OF THE INVENTION

A safety check system of the present invention is designed to comprise a safety detection device that is attached to a body of a checked person and detects her or his safety, a home server for accumulating various pieces of information from the safety detection device, and a terminal of a checker for executing a safety check of the checked person.

Then the safety detection device is designed to comprise a first radio communication mechanism for making a radio communication with the home server, a vital signal detection mechanism for detecting a vital signal of the checked person, and a first control mechanism for creating safety information of the checked person including a presence or absence of the vital signal; the home server is designed to comprise a second radio communication mechanism for making a radio communication with the safety detection device, a detection history memory mechanism for memorizing a detection history of the checked person sequentially in time including the safety information acquired from the safety detection device, and a second control mechanism for sending a detection history of the checked person in the detection history memory mechanism to the terminal when the home server requests the safety detection device for an acquisition of the safety information according to a request from the terminal and does not acquire the vital signal indicating that the checked person lives.

Meanwhile, other configurations will be described in an embodiment of the present invention described later.

### BRIEF DESCRIPTION OF THE DRAWINGS

FIG. 1 is a drawing showing an outline of a safety check system of an embodiment of the present invention.

FIG. 2 is a block diagram showing a configuration of the safety check system in FIG. 1.

FIG. 3A is a drawing exemplifying a state of a safety detection device of a wrist band type in FIG. 1 having been attached; FIG. 3B is a drawing exemplifying a state of the safety detection device of the wrist band type in FIG. 1 having been detached.

FIG. 4 is a drawing exemplifying a history of event information of a checked person memorized in a detection history memory mechanism of FIG. 1.

FIG. 5 is a flowchart showing a procedure of a home server of FIG. 1 making a detection history.

FIG. 6 is a flowchart showing a procedure of the home server acquiring safety information according to a request from a terminal (checker) in FIG. 1.

FIG. 7 is a flowchart showing a procedure of the home server acquiring the safety information according to a request from the terminal (checker) in FIG. 1.

FIG. 8 is a flowchart showing a procedure of the home server acquiring the safety information according to on the request from the terminal (checker) in FIG. 1.

FIG. 9 is a flowchart showing a processing procedure when the home server requests a detection device (checked person) for a response according to a request from the terminal (checker) in FIG. 1.

### DESCRIPTION OF THE MOST PREFERRED EMBODIMENTS

Here will be described a most preferred embodiment (hereinafter referred to as “embodiment”) for embodying such a safety check system of the present invention in detail, referring to drawings.

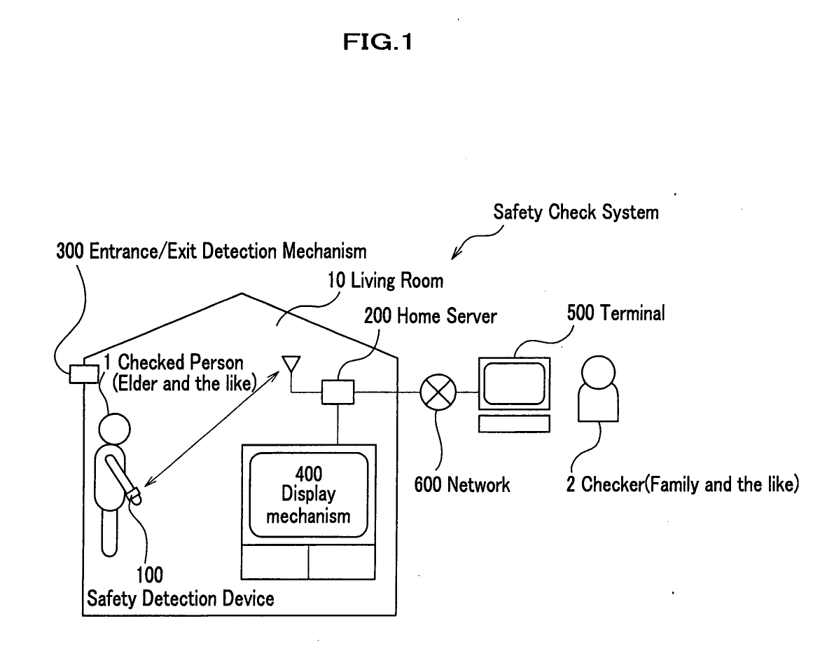

FIG. 1 is a drawing showing an outline of a safety check system of an embodiment of the present invention.

Firstly, the outline of the safety check system of the embodiment will be described.

The safety check system comprises a safety detection device 100 for detecting safety of a checked person 1; a home server 200 for accumulating event information of the checked person 1 sequentially in time, that is, the safety, any of an entrance into and exit out of a living room 10, and the like; an entrance/exit detection mechanism 300 for detecting the entrance/exit of the checked person 1; a display mechanism 400 for displaying a message from the home server 200; a terminal 500 where a checker 2 inputs/outputs various pieces of information; and a network 600 for connecting the home server 200 and the terminal 500.

Meanwhile, the display mechanism 400 is, for example, a television; the terminal 500 is, for example, any of a PC (Personal Computer) and a PDA (Personal Digital Assistant). In addition, the network 600 is, for example, any of an Internet network and a local IP network.

The home server 200 installed in the living room 10 acquires information such as safety information (presence or absence of a vital signal such as a pulse) of the checked person 1 from the safety detection device 100 via an antenna and the like at every predetermined time; information such as an attachment/detachment and battery-running-out of the safety detection device 100; and the like. In addition, the home server 200 also acquires information relating to the entrance/exit of the checked person 1 from the entrance/exit detection mechanism 300 for detecting the entrance into and exit out of the living room 10. Then, the home server 200 memorizes the information (event information) sequentially in time together with occurrence time of the event.

If the home server 200 receives an acquisition request of the safety information of the checked person 1 from the terminal 500 (checker 2), it requests the safety detection device 100 for the acquisition of the safety information.

Here, if the home server 200 acquires the safety information from the safety detection device 100 and can have checked the living of the checked person 1, it returns the above to the terminal 500 (checker 2).

On the other hand, if although the home server 200 acquires the safety information, cannot check the living of the checked person 1, that is, the vital signal of the checked person 1 is not included in the safety information acquired, the server 200 returns the above fact and the newest event information and occurrence time of the newest event out of the event information of the checked person 1 accumulated in the server 200. In addition, if the home server 200 cannot acquire the safety information itself from the safety detection device 100, the server 200 replies to the terminal 500 (checker 2) a reason (communication failure, battery-running-out, or detachment of the safety detection device 100, or exit out of the checked person 1) why the server 200 cannot acquire the information and occurrence time when the reason occurred.

Here, if the home server 200 receives a message (response request) for requesting a direct response check from the terminal 500 (checker 2) to the checked person 1, the server 200 makes the safety detection device 100 or the display mechanism 400 display a message for prompting the checked person 1 to input a response in the device 100.

Here, the message displayed in the display mechanism 400 is one, for example, by any of an image and voice such as “Dear Mr. XXX, your family asks whether you are fine or not. Please push the response input button of the safety detection device.” The message which the safety detection device 100 is made to display (send) is, for example, an alarm by sound, light, vibration, and the like.

Thus even if the family (checker 2) cannot acquire the safety information of the elder (checked person 1) living alone, the home server 200 presents a reason why it cannot acquire the safety information; the newest event information and occurrence time of the newest event; and the like. In addition, because the checker 2 can also directly request the checked person 1 for a response as necessary, it becomes easy for the checker 2 to check the safety of the checked person 1, and the home server 200 can alleviate an anxiety of the checker 2 relating to the safety check.

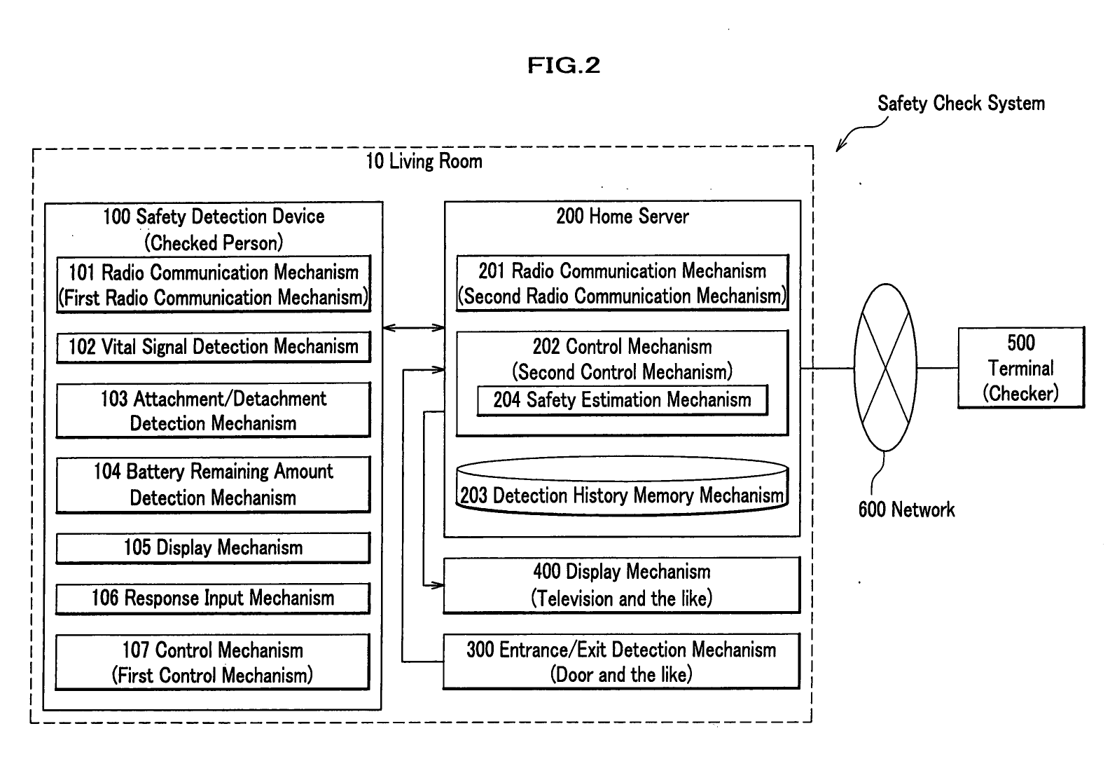

Next, using FIG. 2, each component of the safety check system of the embodiment will be described in detail. FIG. 2 is a block diagram showing a configuration of the safety check system in FIG. 1.

Firstly, the safety detection device 100 will be described.

As shown in FIG. 2, the safety detection device 100 comprises a radio communication mechanism 101 (first radio communication mechanism), a vital signal detection mechanism 102, an attachment/detachment detection mechanism 103, a battery remaining amount detection mechanism 104, a display mechanism 105, a response input mechanism 106, and a control mechanism 107 (first control mechanism).

The safety detection device 100 is attached to the checked person 1, detects safety information indicating a presence or absence of a vital signal (presence or absence of such a pulse) of the checked person 1, and sends it to the home server 200 by radio. In addition, the home server 200 detects the attachment/detachment of the safety detection device 100, checks whether or not the battery remaining amount is not more than a predetermined value, and sends the information relating to these to the home server 200.

Furthermore, if the safety detection device 100 receives a request for a response check of the checked person 1 from the home server 200, the device 100 prompts the checked person 1 to input a response therein. Then, if the safety detection device 100 detects the response input of the checked person 1, the device 100 sends it to the home server 200.

The radio communication mechanism 101 is a mechanism by which the safety detection device 100 performs a radio communication with the home server 200, and is realized by a radio antenna and a transmitter/receiver. In addition, the radio communication may also be a communication by infrared ray.

The vital signal detection mechanism 102 is a mechanism for detecting a vital signal (pulse and the like) indicating the living of the checked person 1 and, for example, comprises a pulse sensor and the like. If the vital signal detection mechanism 102 is the mechanism that can detect the vital signal of the checked person 1, it may be any one of a blood pressure sensor, a body temperature sensor, a body movement sensor (acceleration sensor), and the like, and a combination thereof.

The attachment/detachment detection mechanism 103 is a mechanism for detecting the attachment/detachment (whether the safety detection device 100 is attached or detached) of the device 100 of the checked person 1.

Meanwhile, in the embodiment it is assumed that the “attachment” means “attaching the safety detection device 100 to a body,” the “detachment” means “detaching the device 100 from the body,” and the “attachment/detachment” means “attaching the device 100 to the body and detaching it therefrom.”

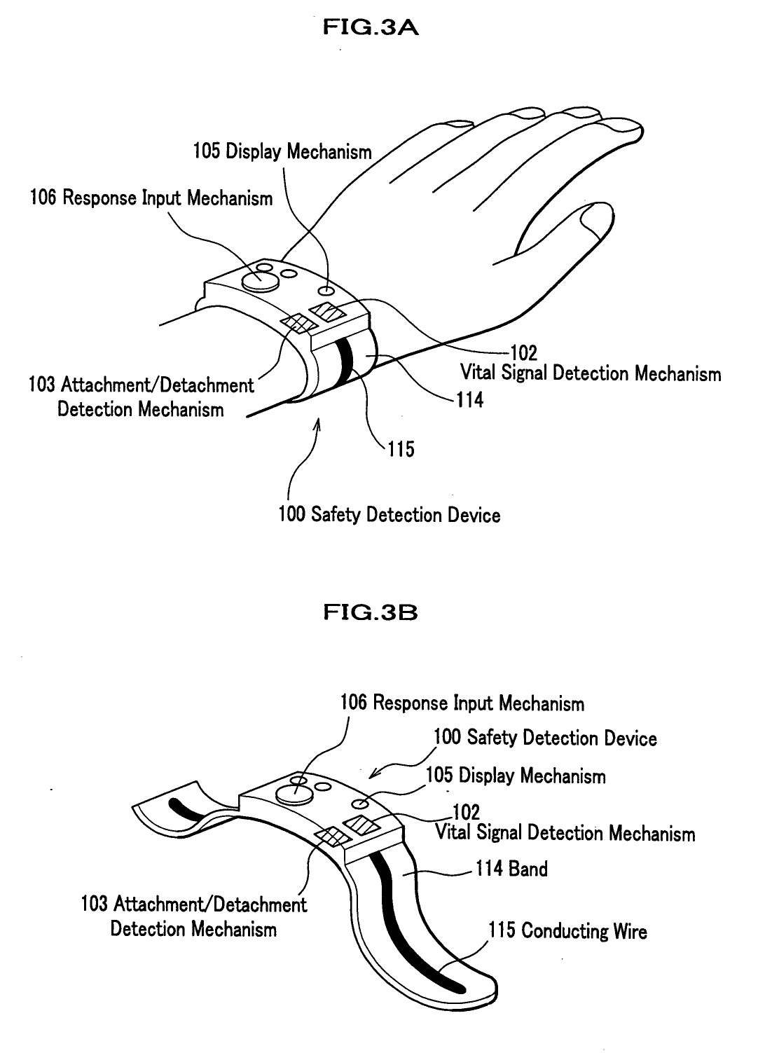

Here, a concrete example of the safety detection device 100 comprising the attachment/detachment detection mechanism 103 is shown in FIGS. 3A and 3B.

FIG. 3A is a drawing exemplifying a state of a safety detection device of a wrist band type in FIG. 1 having been attached; FIG. 3B is a drawing exemplifying a state of the safety detection device of the wrist band type in FIG. 1 having been detached.

In the safety detection device 100 a conducting wire 115 is provided at a band 114, and if the device 100 is attached to a wrist of the checked person 1, a current flows in the conducting wire 115 and the attachment/detachment detection mechanism 103 detects an attachment state; if the device 100 is detached from the wrist, the current of the conducting wire 115 is broken and the device 100 detects a detachment state thereof.

In addition, the attachment/detachment detection mechanism 103 may be designed to be combined with the vital signal detection mechanism 102 (pulse sensor and the like). In other words, the home server 200 may also be designed to determine that the checked person 1 has attached the safety detection device 100 when a vital signal such as a pulse is initially acquired.

Returning to a description of FIG. 2, the battery remaining amount detection mechanism 104 is a mechanism that detects a battery remaining amount of the safety detection device 100, refers to a predetermined threshold memorized in a memory unit not shown, and checks whether or not the battery remaining amount is not more than the predetermined threshold (low battery remaining amount).

The display mechanism 105 is a mechanism that outputs an alarm such as a light, a sound, and a vibration for prompting the checked person 1 to input a response when there exists a response request from the home server 200. In addition, the display mechanism 105 may also comprise a function of displaying the alarm when the battery remaining amount detection mechanism 104 has detected the low battery remaining amount.

The response input mechanism 106 is a mechanism that receives a response input from the checked person 1 and, for example, comprises a button and the like. If a response is input (if the response button is pushed) from the checked person 1, the response input mechanism 106 sends information relating to the response to the home server 200 via the radio communication mechanism 101.

The control mechanism 107 makes the safety detection device 100 execute the detection of the vital signal of the checked person 1 according to a request from the home server 200, and creates safety information including the detection result. Then the control mechanism 107 sends the information to the home server 200 via the radio communication mechanism 101.

In addition, when the attachment/detachment detection mechanism 103 detects the attachment/detachment of the safety detection device 100, and the battery remaining amount detection mechanism 104 detects the low battery remaining amount, the control mechanism 107 has a function of sending these pieces of information to the home server 200 via the radio communication mechanism 101.

Meanwhile, when the control mechanism 107 sends various pieces of information from the safety detection device 100, it may also be designed to send each piece of the information, adding to it a flag indicating a kind (safety information, attachment/detachment information, or low battery remaining amount information) of the each piece of the information and an ID (identification information) of the safety detection device 100. For example, if information sent by the control mechanism 107 is safety information, the mechanism 107 sends it to the home server 200, adding to a content (such a presence or absence of the vital signal) of the safety information a flag indicating that the information is the safety information and the ID of the safety detection device 100.

Furthermore, if when the control mechanism 107 receives an operation check packet for checking an operation of the safety detection device 100 from the home server 200, the operation of the device 100 is operable, the mechanism 107 has a function of sending a reply packet, where own ID of the device 100 is added, to the home server 200. The home server 200 can check whether or not the safety detection device 100 is operable according to the presence or absence of the reply packet.

The control mechanism 107 is assumed to be realized by such a CPU (Central Processing Unit) and a memory, and the CPU is assumed to run a program for the safety detection device 100 memorized in the memory. Meanwhile, the ID of the safety detection device 100 is assumed to be memorized in part of the memory.

Next, the home server 200 will be described.

As described above, the home server 200 acquires such the safety information (presence or absence of a vital signal such a pulse) of the checked person 1 from the safety detection device 100 at every predetermined time; and information such as the attachment/detachment and battery-running-out of the device 100. In addition, the home server 200 also acquires information of the checked person 1 relating to any of an entrance into and exit out of the living room 10. In other words, the home server 200 is a mechanism that memorizes the information (event information) sequentially with time when the information is acquired. The home server 200 comprises a radio communication mechanism 201 (second radio communication mechanism), a control mechanism 202 (second control mechanism), a detection history memory mechanism 203, and a safety estimation mechanism 204.

The radio communication mechanism 201 is a mechanism by which the home server 200 executes a radio communication with the safety detection device 100, and is realized by a radio antenna and a transmitter/receiver. In addition, the radio communication may also be a communication by infrared ray.

The control mechanism 202 executes an acquisition request for safety information at predetermined time referring to a timer not shown or according to a request from the terminal 500. The control mechanism 202 is a mechanism that searches newest (or recent) event information of the checked person 1 from the detection history memory mechanism 203 and sends (presents) the information to the terminal 500.

In addition, the control mechanism 202 has the safety estimation mechanism 204 that estimates a reason why the mechanism 204 cannot estimate a living state of the checked person 1 and acquire the vital signal thereof, and presents the safety estimation result of the checked person 1 of the estimation mechanism 204 and the searched newest event information to the terminal 500 via the network 600.

Meanwhile, at this time the control mechanism 202 may also be designed to combinedly present information relating to occurrence time (occurrence estimation time) of the event. Thus, the checker 2 can grasp more accurate safety information (safety situation) of the checked person 1.

Furthermore, if the control mechanism 202 receives a request (response request) of a response check of the checked person 1 from the terminal 500, it prompts the checked person 1 to input a response in the safety detection device 100. To be more precise, when the checked person 1 is in the living room, the control mechanism 202 makes the display mechanism 400 of the living room 10 display a message (for example, “Dear Mr. XXX, your family asks whether you are fine or not. Please push the button of the safety detection device,” and the like) for prompting her or him to input a response; when the checked person 1 is not in the living room, the control mechanism 202 is designed to send the request of the response check of the checked person 1 to the safety detection device 100 and to make the device 100 display such an alarm in the display mechanism 105.

In addition, when the control mechanism 202 receives any of an acquisition request and response request of the safety information of the checked person 1 from the terminal 500, the mechanism 202 also has a function of authenticating whether or not the request is from a predetermined checker 2 (for example, family). Because the control mechanism 202 has such the function, a security level can be enhanced in presenting a safety check result.

Meanwhile, the control mechanism 202 is assumed to be realized by such a CPU and a memory, and the CPU is assumed to read a program (that is, safety check program), which runs a safety check method of the embodiment, onto the memory from a not shown memory unit of the home server 200, and to run the program.

If the detection history memory mechanism 203 acquires various pieces of event information of the checked person 1, it memorizes and accumulates them sequentially in time. In other words, the detection history memory mechanism 203 is a mechanism that stores a detection history that memorizes the event information sequentially in time. In addition, the event information may be designed to be memorized in order other a time sequence. For example, a higher order or a lower order in importance of the content of the event information is also available.

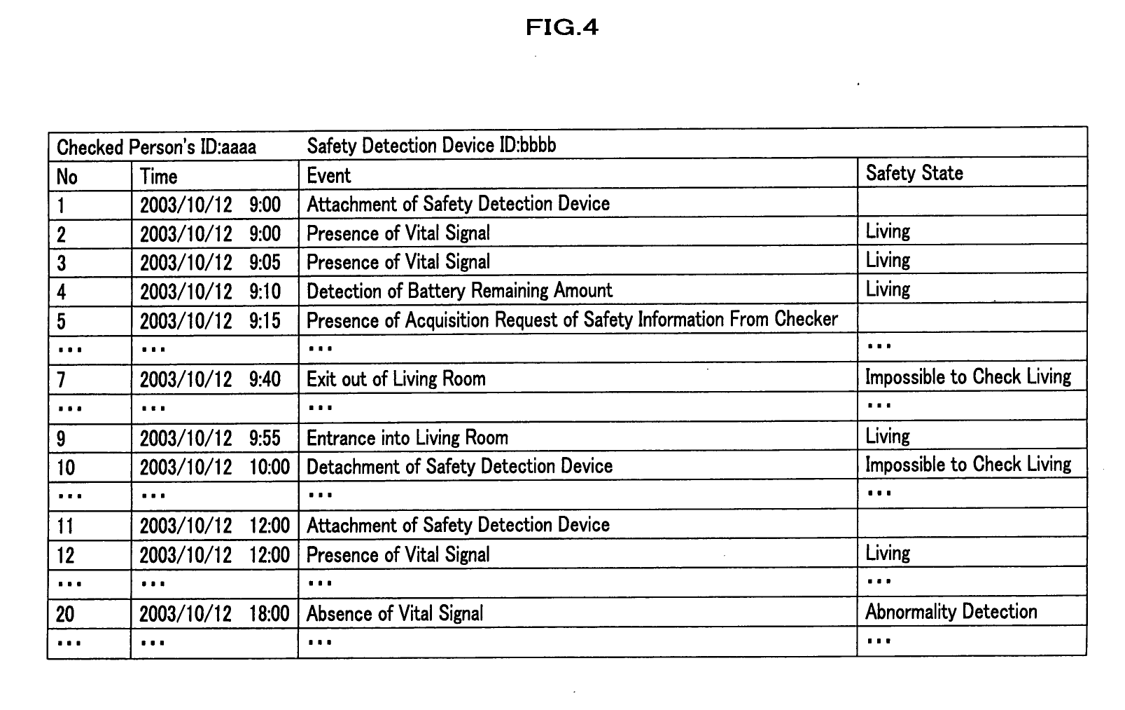

FIG. 4 is a drawing exemplifying a history (detection history) of event information of a checked person memorized in a detection history memory mechanism.

For example, as exemplified in FIG. 4, the detection history comprises an ID (identification information) of the checked person 1, an ID of the safety detection device 100, a serial number (No) of each event, occurrence time of the event, a content of the event, and an item of a safety state of the checked person 1.

The detection history in FIG. 4 is the history of the ID of the checked person 1, aaaa and the ID of the safety detection device 100, bbbb; and No 1 information indicates that the safety detection device 100 is attached at 9:00, Oct. 12, 2003. No 2 information indicates that the safety detection device 100 acquires a vital signal of the checked person 1 at 9:00, Oct. 12, 2003 and that she or he is estimated to live.

Meanwhile, the occurrence time of each event may be any time when the safety detection device 100 detected the event or the home server 200 acquired the event information.

The content of the event is, for example, an attachment/detachment of the safety detection device 100, a presence or absence of the vital signal of the checked person 1, a detection of the low battery remaining amount, a presence or absence of the acquisition request of the safety information of the checked person 1 from the checker 2, and the like.

The safety state of the checked person 1 is, for example, a living state, an abnormality detection, an uncheckable state, an exit state, and the like. The safety estimation mechanism 204 executes the estimation of the safety by referring to the content of the event information. A determination logic of the safety estimation mechanism 204 at this time will be described, using Table 1.

Table 1 is a table where the determination logic of the safety state of the safety estimation mechanism 204 is exemplified. As shown in Table 1, referring to the event information, when the safety estimation mechanism 204 determines that the checked person 1 is in the living room 10, has attached the safety detection device 100, and has a vital signal (pulse and the like), the mechanism 204 estimates that she or he “lives.” In addition, when there exists no vital signal in spite of her or his having attached the safety detection device 100, the safety estimation mechanism 204 estimates it as the “abnormality detection”; when she or he has not attached the device 100, the mechanism 204 assumes it as the “uncheckable state.” Then the safety estimation mechanism 204 writes the estimation result in the column of the safety state of the detection history.

Meanwhile, when the checked person 1 has exited out of the living room 10, the safety estimation mechanism 204 estimates it as the “Outing state,” not depending on any of the attachment/detachment of the safety detection device 100 and the presence or absence of the vital signal; or makes it the “uncheckable state.”

TABLE 1
Determination Logic of Safety State

| Entrance into | | Presence or | |
|---|---|---|---|
| and Exit out of | Attachment/Detachment of | Absence of Vital | |
| Living Room | Safety Detection Device | Signal | Safety State |
| Entrance | Attachment | Presence of Vital | Living |
| | | Signal | |
| Entrance | Attachment | Absence of Vital | Abnormality |
| | | Signal | Detection |
| Entrance | Detachment | — | Uncheckable |
| | | | State |
| Exit | — | — | Outing |
| | | | (Uncheckable |
| | | | State) |

Here, if when the vital signal detection mechanism 102 of the safety detection device 100 is configured of a combination of a plurality of sensors, a vital signal exists in any one of the sensors and the checked person 1 has attached the device 100, the safety estimation mechanism 204 may also be designed to estimate it as the “living.”

Meanwhile, when the control mechanism 202 presents event information searched from the detection history memory mechanism 203 to the terminal 500, the mechanism 202 may be designed to present one of newest event information of the checked person 1 or plural pieces thereof sequentially in time. In a case of presenting the plural pieces, the control mechanism 202 may also be designed to present them, cutting out a period (for example, within past two weeks) designated by the checker 2, or the detection history itself. Thus if the control mechanism 202 is designed to present the event information of the checked person 1 by the plural pieces sequentially in time, the checker 2 can grasp a recent safety state of the checked person 1 more in detail. In addition, the control mechanism 202 may also be designed to determine the content of the event information and to preferentially present more important event information.

The entrance/exit detection mechanism 300 is a mechanism that detects the checked person 1, who has attached the safety detection device 100, to enter into and exit out of the living room 10, and that sends the detection result to the home server 200.

The entrance/exit detection mechanism 300 is realized, for example, by sensors that is installed at outside and inside of an entrance/exit door of the living room 10 and reads the ID of the safety detection device 100; a radio antenna; and a CPU. In other words, installing a sensor A (outside) and sensor B (inside) for reading the ID of the checked person 1 at the outside and inside of the door of the living room 10, respectively, in advance, the sensors reads the ID via the radio antenna, and the entrance/exit detection mechanism 300 determines an order in which the CPU has read the ID, and detects the entrance/exit of the checked person 1. In other words, in a case of the sensor A (outside) having read and then the sensor B (inside) in this order, the entrance/exit detection mechanism 300 is designed to detect an “entrance of the checked person 1.” Whereas in a case of the sensor B having read (inside) and then the sensor A (outside) in this order, the entrance/exit detection mechanism 300 is designed to detect an “exit of the checked person 1.” Then every time when the entrance/exit detection mechanism 300 detects the checked person 1 to enter into and exit out of the living room 10, it sends the detection result to the home server 200.

Meanwhile, also when the entrance/exit detection mechanism 300 sends the information of the entrance/exit to the home server 200, it may be designed to send the information, adding to it a flag indicating a kind thereof, the ID of the safety detection device 100 (or living room 10), and the like. Thus the home server 200 can check entrances/exits of a plurality of living rooms 10.

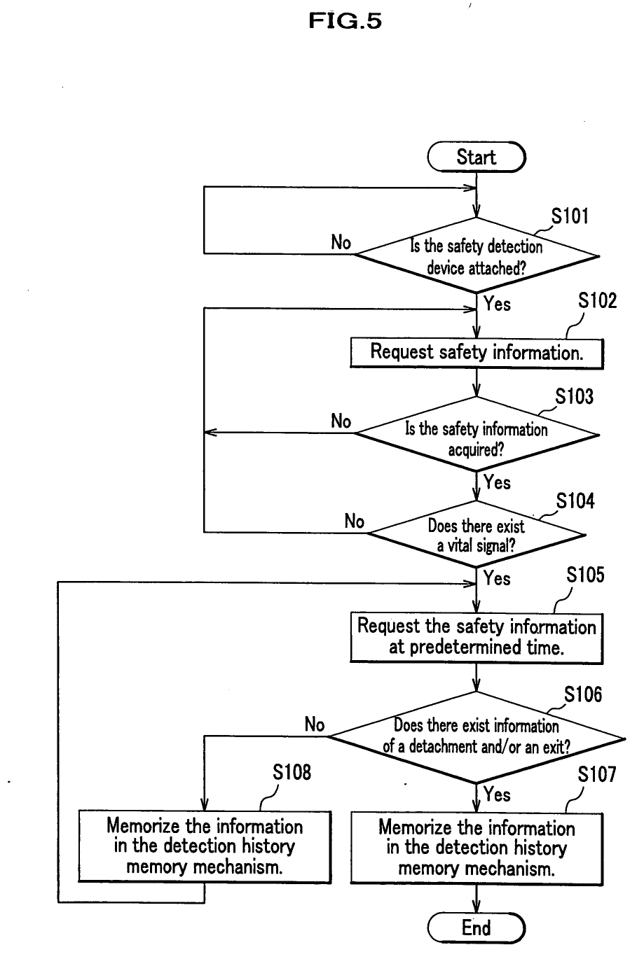

Next, an operation of each component of the safety detection device 100 will be described in detail, using FIGS. 5 to 8 with referring to FIGS. 1 to 4 as needed.

FIG. 5 is a flowchart showing a procedure of a home server making a detection history.

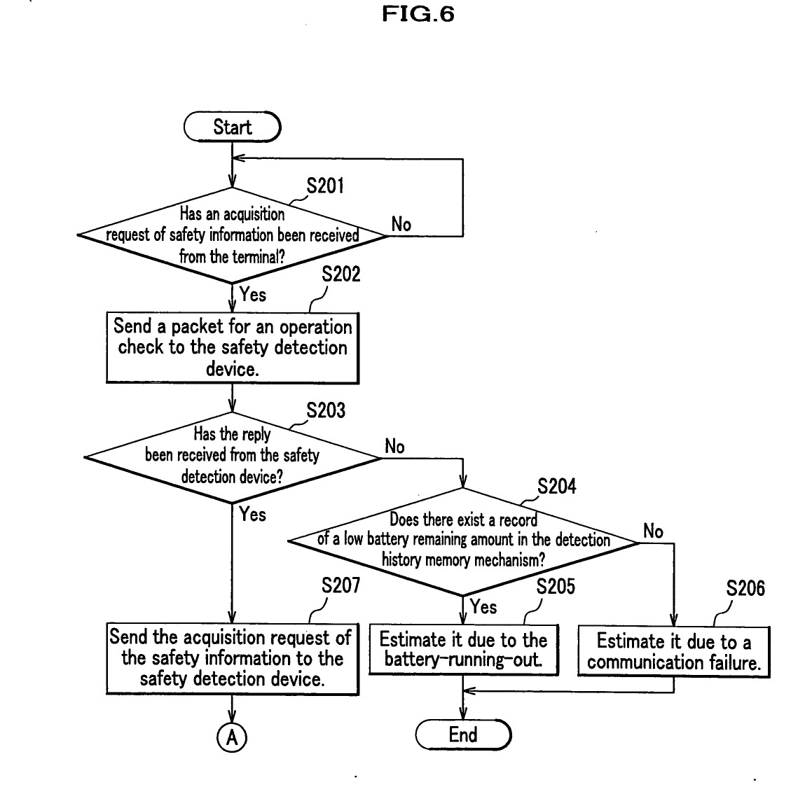

FIGS. 6 to 8 are flowcharts showing procedures of the home server acquiring safety information according to a request from a terminal (checker).

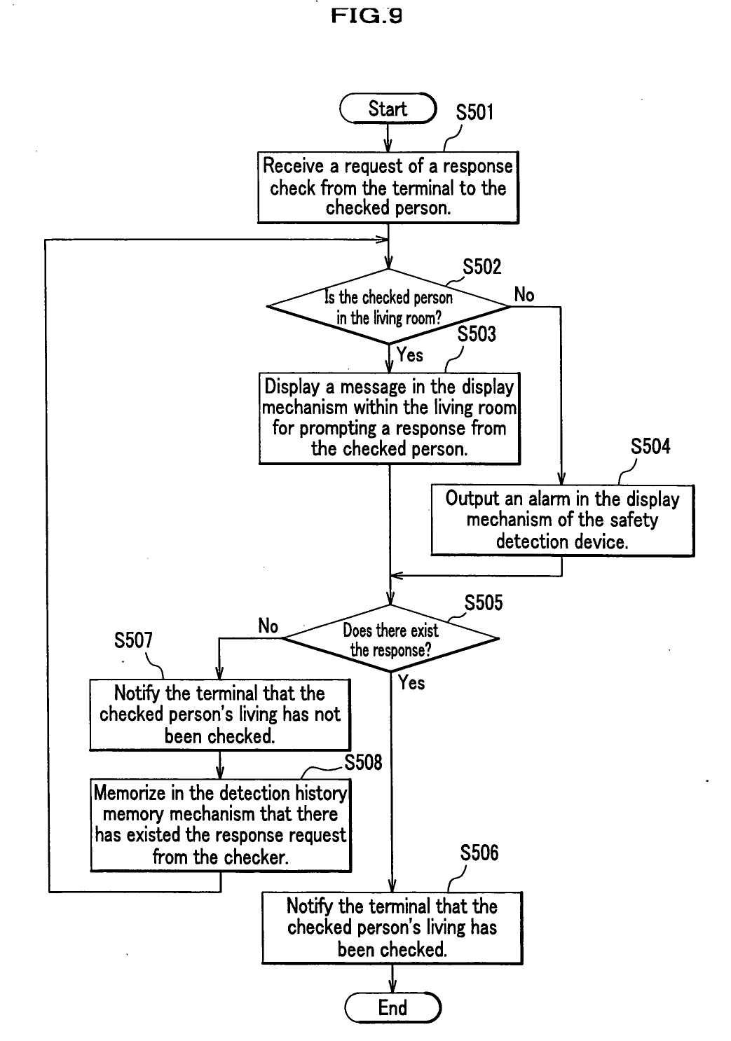

FIG. 9 is a flowchart showing a processing procedure when the home server requests a safety detection device (checked person) for a response according to a request from the terminal (checker).

When the home server 200 detects an attachment of the safety detection device 100, it starts to acquire safety information and accumulate a detection history; when the home server 200 detects a detachment of the safety detection device 100 or that that checked person 1 has exited out of the living room 10, it ends to acquire the safety information and accumulate the detection history.

The processing procedure of the home server 200 will be described, using FIG. 5 with referring to FIGS. 1 to 4 as needed.

[Creation Procedure of Detection History]

Firstly, if the home server 200 receives event information that the checked person 1 has attached the safety detection device 100 (Yes in step S101), the control mechanism 202 of the home server 200 requests the device 100 for an acquisition of safety information via the radio communication mechanism 201 (step S102).

Next, if after the acquisition in the step S102 the home server 200 acquires the safety information from the safety detection device 100 within predetermined time (Yes in step S103), it determines whether or not a vital signal exists in the safety information (step S104).

Meanwhile, when the home server 200 cannot acquire the safety information from the safety detection device 100 even after not less than predetermined time (No in the step S103), the server 200 returns to the step S102 and again requests the acquisition of the safety information.

When the vital signal exists in the safety information acquired in the step S104 (Yes in the step S104), the control mechanism 202 requests the safety detection device 100 for the acquisition of the safety information at every predetermined time (for example, five minutes) (step S105). Then if the home server 200 acquires the safety information, it memorizes (accumulates) the information in the detection history memory mechanism 203 together with time when the information is acquired. Here, when the vital signal does not exist in the safety information acquired (No in the step S104), the home server 200 returns to the step S102 and again requests the safety detection device 100 for the acquisition of the safety information.

Next, the control mechanism 202 of the home server 200 checks the flag of the acquired event information of the checked person 1 and determines the information (step S106). To be more precise, the control mechanism 202 determines whether the acquired information is one relating to the detachment of the safety detection device 100 and/or the exit, or other than these. Here, the information acquired by the home server 200 is one indicating the detachment of the safety detection device 100 or the exit out of the living room 10 (detachment and/or exit in the step S106), the home server 200 memorizes (step S107) the information in the detection history memory mechanism 203 together with the time when the information is acquired, and ends the procedure. In other words, if the checked person 1 detaches the safety detection device 100 and/or exits out of the living room 10, the home server 200 memorizes it in the detection history memory mechanism 203 and once ends to acquire the safety information (memorize the detection history).

On the other hand, when the information acquired by the home server 200 is one indicating other than the detachment of the safety detection device 100 and/or the exit (other than any of the detachment and the exit in the step S106), the home server 200 memorizes (step S108) the information and the time, when the information is acquired, in the detection history memory mechanism 203 and returns to the processing in the step S105.

Thus, the home server 200 is designed to accumulate the event information of the checked person 1 and to create the detection history, and thereby, the checker 2 can grasp a recent (newest) safety situation of the checked person 1 by referring to the detection history even when her or his safety information cannot be acquired, and when her or his response cannot be acquired. In addition, because the home server 200 is designed to acquire the safety information only when the checked person 1 attaches the safety detection device 100 and is in the living room 10, and not to acquire the safety information other than this, the server 200 can reduce the consumption of the battery of the device 100. In other words, the home server 200 can lower a possibility of becoming unable to check the safety of the checked person 1 due to the battery-running-out of the safety detection device 100.

[Acquisition Procedure of Safety Information Based on Request from Checker]

Next, a processing procedure will be described when the home server 200 acquires safety information, based on a request from the terminal 500 (checker 2), using FIGS. 6 to 8 with referring to FIGS. 1 to 4 as needed.

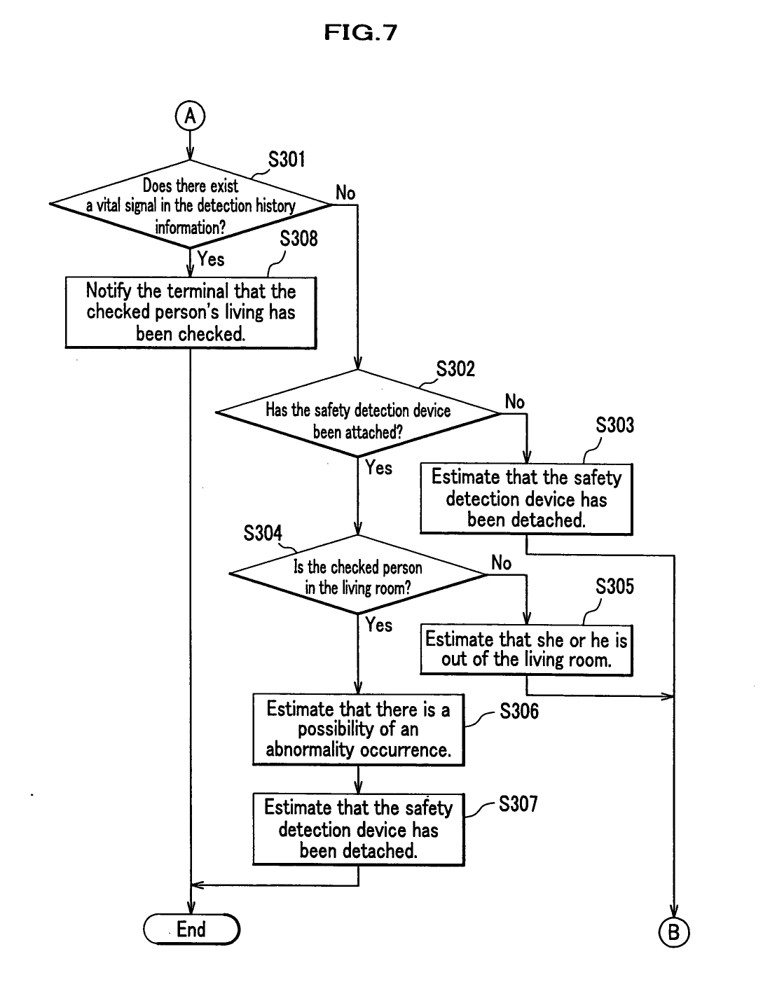

Firstly, if the home server 200 receives an acquisition request of the safety information from the terminal 500 (Yes in step S201), it executes a predetermined authentication for the terminal 500 and checks that the acquisition request is surely from the checker 2. Then if the home server 200 can check that the acquisition request of the safety information is from the checker 2, the control mechanism 202 sends an operation check packet in order to check whether or not the safety detection device 100 can send the safety information (step S202). Here, if the control mechanism 202 receives a reply of the operation check packet from the safety detection device 100 (Yes in step S203), the home server 200 sends the acquisition request of the safety information to the device 100 (step S207) and proceeds to step S301 in FIG. 7.

On the other hand, if the home server 200 cannot receive the reply of the operation check packet from the safety detection device 100 (No in the step S203), the safety estimation mechanism 204 checks whether or not there exists a record of the low battery remaining amount in the detection history of the checked person 1 of the detection history memory mechanism 203 (step S204). Then in a case that there exists the record of the low battery remaining amount in the detection history (Yes in the step S204), the safety estimation mechanism 204 estimates that the battery-running-out has occurred in the safety detection device 100. In other words, the safety estimation mechanism 204 estimates that a reason why there is no response (reply) from the safety detection device 100 is due to the battery-running-out. Then the safety estimation mechanism 204 presents (sends) (step S205) to the terminal 500, for example, such a message as “The safety information of Mr. XXX cannot have been acquired. There is a possibility of the battery of the safety detection device having run out.” and ends the processing.

In this connection, at this time the control mechanism 202 may also be designed to combinedly present newest event information out of event information memorized in the detection history memory mechanism 203 and occurrence time of the event. In addition, the determination of whether or not there exists the record of the low battery remaining amount in the detection history may also be designed to be performed by where or not there exists the record of the low battery remaining amount within a predetermined period (for example, several weeks) from a current date.

Then, in a case that there exists no record of the low battery remaining amount in the detection history (no in the step S204), the safety estimation mechanism 204 estimates that a communication failure has occurred in the safety detection device 100. In other words, the home server 200 estimates that a reason why there is no response (reply) from the safety detection device 100 is due to a communication failure. Then the home server 200 presents (sends) a message such as “The safety information of Mr. XXX cannot have been acquired. She or he may be at a place where an electric wave does not reach or there is a possibility of a communication failure.” to the terminal 500 (step S206) and ends the processing.

Meanwhile, when the safety estimation mechanism 204 presents the estimation result to the terminal 500, the control mechanism 202 may also be designed to combinedly present newest event information of the checked person 1 and occurrence time of the event.

Thus, when the home server 200 cannot acquire the response of the operation check packet from the safety detection device 100, the safety estimation mechanism 204 estimates the reason (battery-running-out and communication failure) why the operation check packet is not responded from the detection history of the checked person 1, presents the estimation result to the terminal 500 (checker 2), and thereby, can alleviate an anxiety relating to the safety check of the checker 2.

Meanwhile, although when in the procedure the home server 200 requests an acquisition of the safety information, it is designed to send the operation check packet to the safety detection device 100 in advance, it may also be designed to directly request the acquisition of the safety information. In other words, it may also be designed to determine whether or not the safety detection device 100 operates by whether or not the home server 200 can have acquired the safety information.

Removing to a description of FIG. 7, in a case that there exists the vital signal in the safety information that the home server 200 has acquired from the safety detection device 100 (Yes in step S301), the home server 200 presents a message such as “It has been checked that Mr. XXX is fine at 9:30, Sept. 20, 2003.” to the terminal 500 (checker 2), notifies it that the living of the checked person 1 can have been checked (step S308), and ends the processing.

On the other hand, when the control mechanism 202 determines that there exists no vital signal in the safety information acquired (No in the step S301), refers to the detection history of the checked person 1 of the detection history memory mechanism 203, and determines that the checked person 1 has attached the safety detection device 100 (Yes in step S302), the mechanism 202 refers to the detection history of the checked person 1 and determines (estimates) whether or not the checked person 1 is in the living room 10 (step S304)

Here, when the control mechanism 202 determines whether or not the checked person 1 is in the living room 10 (that is, when although it is estimated that the checked person 1 is in the living room 10, there exists no her or his vital signal.) (Yes in the step S304), the mechanism 202 estimates that “there is a possibility of an abnormality occurrence” (step S306). Then the control mechanism 202 presents (sends) the estimation result and the newest event information of the checked person 1 and the occurrence time of the event searched by the detection history memory mechanism 203, for example, with a message such as “The safety information of Mr. XXX cannot have been acquired. It was ten minutes before when the safety information could be lastly acquired.” to the terminal 500 (step S307).

Meanwhile, here, the control mechanism 202 may also be designed to present a message for checking the checker 2 whether or not the abnormality detection of the checked person 1 may be notified to an administrator such as “May you notify the administrator of the abnormality detection?” to the terminal 500.

Thus the home server 200 estimates the safety of the checked person 1 and sends the newest event information to the terminal 500 (checker 2), and thereby the checker 2 can quickly grasp that some abnormality (falling-down in the living room 10 and the like) has occurred in the checked person 1.

In addition, because the information includes the occurrence time of each piece of event information, it becomes easier for the checker 2 to estimate how long the checked person 1 has fallen down and the like. Accordingly, the home server 200 can alleviate an anxiety relating to the safety check of the checker 2 (or administrator of the living room 10) and can also speedily execute a care in the abnormality occurrence.

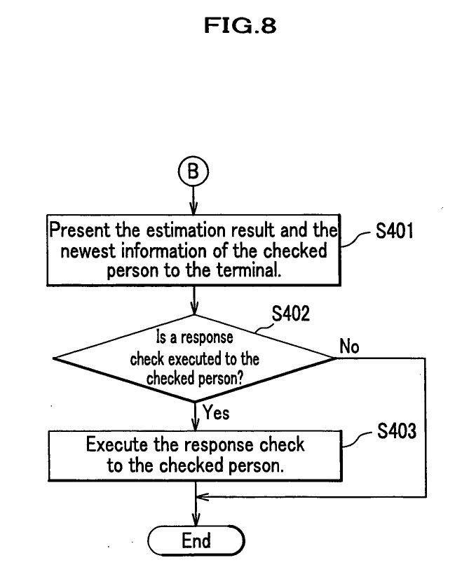

Meanwhile, in the step S302 of the case of no vital signal existing in the checked person 1, when in the detection history there exists no record that the checked person 1 attached the safety detection device 100, the control mechanism 202 estimates that she or he has detached the device 100 (step S303), and presents (sends) the estimation result to the terminal 500 with a message such as “Mr. XXX has not attached the safety detection device” (step S401 in FIG. 8). At this time the control mechanism 202 also presents the terminal 500 a message (such as “May you request Mr. XXX to respond to you?”) for asking whether or not the terminal 500 requests the checked person 1 for a response check.

In addition, in the step S304 where there exists no vital signal in the checked person 1 and she or he is under the attachment of the safety detection device 100, when there does not exists her or his entrance record in the detection history (No in the step S304) the control mechanism 202 estimates that the checked person 1 is out (not in the living room 10) (step S305), the control mechanism 202 presents (sends) a message such as “Mr. XXX is now out.” to the terminal 500. In addition, the control mechanism 202 also presents the terminal 500 a message for asking whether or not the terminal 500 requests the checked person 1 for a response check (step S401).

Here, if the terminal 500 (checker 2) receives an input of requesting the checked person 1 for the response check and the home server 200 receives the information (Yes in step S402), the server 200 checks the response for the checked person 1 (step S403) and ends the processing.

On the other hand, if the home server 200 does not receive the information of requesting the checked person 1 for the response check from the terminal 500 (checker 2) (No in the step S402), that is, when the home server 200 receives the information of no necessity existing in requesting her or him for the response check from the terminal 500 or does not receive the information of necessity existing in requesting the response check, the server 200 ends the processing as it is.

[Procedure of Response Check]

Next, a processing procedure in the home server 200 executing (requesting) a response check for the checked person 1 will be described in detail, using FIG. 9 with referring to FIGS. 1 to 4 as needed.

Firstly, if the home server 200 receives a message for requesting the checked person 1 for the response check (response request) (step S501), the safety estimation mechanism 204 refers to the detection history of the checked person 1 of the detection history memory mechanism 203 and determines (estimates) whether or not the checked person 1 is in the living room 10 (step S502). Here, if the safety estimation mechanism 204 determines (estimates) that the checked person 1 is in the living room 10 (Yes in the step S502), it makes the display mechanism 400 (television and the like) in the living room 10 a message for prompting the checked person 1 to input a response (for example, a message such as “Mr. XXX, your family asks whether or not you are fine. Please push the response input button of the safety detection device.” (step S503).

On the other hand, if the safety estimation mechanism 204 refers to the detection history of the checked person 1 of the detection history memory mechanism 203 and determines (estimates) that the checked person 1 is not in the living room 10 (No in the step S502), the mechanism 204 estimates that the checked person 1 is out of the living room 10 and outputs an alarm in the display mechanism 105 of the safety detection device 100 (step S504).

Here, if there is the response from the checked person 1 within predetermined time (for example, ten minutes) (Yes in step S505), to be more precise, if the home server 200 detects that the response is input in the response input mechanism 106 of the safety detection device 100, the safety estimation mechanism 204 determines (estimates) that the checked person 1 lives. Then the safety estimation mechanism 204 notifies the terminal 500 (checker 2) of the estimation result with a message such as “It could be checked that Mr. XXX lived at 9:30, Sep. 20, 2003” and ends the processing.

On the other hand, if the safety estimation mechanism 204 cannot acquire the response from the checked person 1 even after the lapse of predetermined time (for example, ten minutes) (No in the step S505), the mechanism 204 notifies the terminal 500 (checker 2) of a message such as “It could not be checked that Mr. XXX lived at 9:30, Sep. 20, 2003” (step S507). Then the safety estimation mechanism 204 memories it also in the detection history memory mechanism 203 (step S508), returns to the step S502 after the elapse of predetermined time, and again requests the safety detection device 100 (checked person 1) for the response check.

Thus, the checker 2 can execute the safety check (response check) by the direct response input from the checked person 1. In addition, because the home server 200 changes a method of the response request by whether or not the checked person 1 is in the living room 10, it can surely execute the response request to her or him.

Thus the safety check system of the embodiment of the present invention has been described. The safety check system is made the configuration thus described, and thereby, the checker 2 (family) can check the safety of the checked person 1 (elder living alone) at arbitrary time. In addition, even if the safety cannot be checked, the checker 2 can grasp a reason why she or he cannot check the safety of the checked person 1, and the recent (newest) safety of the checked person 1. Furthermore, because the checker 2 can grasp approximately when the event occurred, she or he can alleviate an anxiety relating to the safety of the checked person 1.

The present invention is not limited to the embodiment and is changeable without departing from the spirit and scope of the invention. For example, although in the embodiment the descriptions are made assuming that a range (monitoring range) for the home server 200 acquiring the safety information from the safety detection device 100 is in the living room 10, the monitoring range may also be extended to a neighbor thereof. In addition, the home server 200 may also be designed to monitor a plurality of living rooms 10 (checked persons 1, entrance/exit detection mechanisms 300). At this time, by designing to add IDs of the safety detection devices 100 and the living rooms 10 to event information, it may be designed to identify the information for each safety detection device 100 or living room 10.

Thus using a system that can monitor the plurality of the living rooms 10, it also becomes easier to introduce the system into such collective housings where there are many elders living alone.

Meanwhile, as the object of the safety check can be considered a child separately living from her or his family, a handicapped person, and the like other than an elder living alone.

In addition, although a form of the safety detection device 100 is described, making a wrist band type an example thereof, it may also be any of a ring type and a pendant type if the vital signal of the checked person 1 can be acquired.

Meanwhile, the home server 200 related to the embodiment of the present invention can be realized by a computer system comprising a computer and a program, and be provided by a memory medium and by memorizing the program in the memory medium that the computer can read. In addition, it is also enabled to provide the program via a network.

A computer system mentioned here is one that includes a software such as an OS (Operating System) and a hardware such as peripheral equipment.

1. A safety check system of the present invention comprising:
a safety detection device that is attached to a body of a checked person and detects safety of said checked person;
a server for accumulating various pieces of information from said safety detection device; and
a terminal of a checker for executing a safety check of said checked person,
wherein said safety detection device comprises a first radio communication mechanism for making a radio communication with said server, a vital signal detection mechanism for detecting a vital signal of said checked person, and a first control mechanism for creating safety information of said checked person including a presence or absence of said vital signal;
wherein said server comprises a second radio communication mechanism for making a radio communication with said safety detection device, a detection history memory mechanism for memorizing a detection history of said checked person sequentially in time including said safety information acquired from said safety detection device, and a second control mechanism for sending a detection history of said checked person in said detection history memory mechanism to said terminal when said server requests said safety detection device for an acquisition of said safety information according to a request from said terminal and does not acquire said vital signal indicating that said checked person lives.

2. The safety check system according to claim 1, wherein said second control mechanism requests said safety detection device for an acquisition of said safety information according to a request from said terminal and does not acquire said vital signal, said second control mechanism presents newest event information to said terminal from a detection history of said checked person of said detection history memory mechanism.

3. The safety check system according to claim 1,
wherein said safety detection device further comprises at least one of an attachment/detachment detection mechanism for detecting an attachment/detachment of said safety detection device of said checked person and a battery remaining amount detection mechanism for detecting that a battery remaining amount of said safety detection device is not more than a predetermined value,
wherein event information memorized in said detection history memory mechanism further comprises at least one of information relating to the attachment/detachment of said safety detection device and information relating to the battery remaining amount.

4. The safety check system according to claim 1 which detects an entrance and exit of said checked person into and out of a living room and has an entrance/exit detection mechanism for sending information relating to the entrance and the exit, wherein event information memorized in said detection history memory mechanism further comprises the information relating to said entrance and exit.

5. The safety check system according to claim 1, wherein when said second control mechanism does not acquire said vital signal from said safety detection device, said second control mechanism refers to a detection history of said checked person of said detection history memory mechanism, estimates a reason why said second control mechanism does not acquire said vital signal from said safety detection device, and further presents the estimation result to said terminal.

6. A safety check method of using a server for accumulating event information of a checked person composed of at least one of safety information of a checked person detected from a safety detection device for checking safety of the checked person, information relating to an attachment and detachment of said safety detection device, information relating to a battery remaining amount of said safety detection device, and information relating to an entrance and exit into and out of a living room of said checked person, the server comprising the steps of:
acquiring said event information of said checked person;
creating a detection history where said event information memorized sequentially in time;
presenting a detection history of said checked person of said detection history memory mechanism to a terminal when said server requests said safety detection device for an acquisition of said safety information according to a request from said terminal and does not acquire said safety information said checked person.

7. The safety check method according to claim 6, wherein when said server requests said safety detection device for an acquisition of said safety information and does not acquire said safety information from said safety detection device, said server sends newest event information out of the detection history of said checked person of said detection history memory mechanism to said terminal.

8. The safety check method according to claim 6, wherein when said server requests said safety detection device for an acquisition of said safety information and does not acquire said safety information from said safety detection device, said server refers to the detection history of said checked person of said detection history memory mechanism, estimates a reason why said server does not acquire said safety information from said safety detection device, and sends the estimation result to said terminal.

9. A program for checking safety which makes a computer execute a safety check method of using a server for accumulating event information of a checked person composed of at least one of safety information of a checked person detected from a safety detection device for checking safety of the checked person, information relating to an attachment and detachment of said safety detection device, information relating to a battery remaining amount of said safety detection device, and information relating to an entrance and exit into and out of a living room of said checked person, the server comprising the steps of: acquiring said event information of said checked person; creating a detection history where said event information memorized sequentially in time; presenting a detection history of said checked person of said detection history memory mechanism to a terminal when said server requests said safety detection device for an acquisition of said safety information according to a request from said terminal and does not acquire said safety information said checked person.

10. A memory medium read by a computer which memorizes a program for checking safety which makes a computer execute a safety check method of using a server for accumulating event information of a checked person composed of at least one of safety information of a checked person detected from a safety detection device for checking safety of the checked person, information relating to an attachment and detachment of said safety detection device, information relating to a battery remaining amount of said safety detection device, and information relating to an entrance and exit into and out of a living room of said checked person, the server comprising the steps of: acquiring said event information of said checked person; creating a detection history where said event information memorized sequentially in time: presenting a detection history of said checked person of said detection history memory mechanism to a terminal when said server requests said safety detection device for an acquisition of said safety information according to a request from said terminal and does not acquire said safety information said checked person.
